(19) United States
(12) Patent Application Publication
(10) Pub. No.: US 2026/0053789 A1
(43) Pub. Date: Feb. 26, 2026

(54) TREATING ELEVATED INTRAOCULAR PRESSURE WITH NICOTINAMIDE

(71) Applicant: The Trustees of Columbia University in the City of New York, New York, NY (US)

(72) Inventors: Simon W.M. JOHN, Bar Harbor, ME (US); Nicholas TOLMAN, New York, NY (US)

(21) Appl. No.: 19/370,496

(22) Filed: Oct. 27, 2025

Related U.S. Application Data
(63) Continuation-in-part of application No. PCT/US2024/030076, filed on May 17, 2024.
(60) Provisional application No. 63/503,352, filed on May 19, 2023.
(60) Provisional application No. 63/712,251, filed on Oct. 25, 2024.

(51) Int. Cl. A61K 31/455; A61K 31/22; A61K 31/706; A61K 31/7084; A61P 27/02

(52) U.S. Cl. CPC A61K 31/455; A61K 31/22; A61K 31/706; A61K 31/7084; A61P 27/02

(57) ABSTRACT

Methods of treating or preventing elevated intraocular pressure in a subject are disclosed, the methods including administering to the subject a therapeutically effective amount of nicotinamide or a derivative or analog of nicotinamide, or a pharmaceutical composition thereof.

### CROSS REFERENCE TO RELATED APPLICATIONS

This application claims the benefit of and priority to Provisional Application No. 63/712,251, filed on Oct. 25, 2024 and this application is a continuation-in-part of International Patent Application No. PCT/US2024/030076, filed on May 17, 2024, which claims the benefit of and priority to U.S. Provisional Application No. 63/503,352, filed on May 19, 2023, the content of each which is hereby incorporated by reference in their entireties.

### STATEMENT REGARDING FEDERALLY SPONSORED RESEARCH OR DEVELOPMENT

This invention was made with government support under EY032507 awarded by the National Institutes of Health. The government has certain rights in the invention.

### INCORPORATION BY REFERENCE

Any patent, patent publication, journal publication, or other document cited herein is expressly incorporated herein by reference in its entirety.

### FIELD

This application is generally related to methods of treating and/or preventing elevated intraocular pressure (“IOP”) in a subject by administering nicotinamide (“NAM”) to the subject.

### BACKGROUND

Increased intraocular pressure is an important risk factor for glaucoma. However, elevated IOP alone is not glaucoma. Ocular hypertensives, who have high IOP but not glaucoma, could benefit from IOP lowering. Existing treatments are typically applied only after detecting high IOP. Many currently used IOP treatments are delivered topically to the eye and can be difficult and time-consuming for patients to apply and can be poorly tolerated. Patients can suffer from systemic effects such as hypotension, arrhythmias, bronchospasm, lightheadedness, and somnolence. Even the newer and typical first-line treatments, prostaglandin analogs, are reported to cause a variety of side effects including hyperemia, ocular irritation, ocular pain, foreign body sensation, darkening of eyelid skin, patchy darkening of iris color, abnormal eyelash growth, corneal thinning, and visual blur. Additionally, more than half of patients on topical anti-hypertensive medications have ocular surface disease, and allergic reactions to topical therapy are common. There are no current treatments that prevent IOP from becoming elevated.

While topical monotherapy is the preferred treatment for IOP control in accordance with European Glaucoma Society Terminology and Guidelines, most patients require multidrug therapy to achieve their target IOPs. These medications are associated with significant financial costs, especially as patients need to chronically use these therapies until they undergo surgical intervention for IOP lowering.

Methods for treating or preventing IOP from becoming elevated would be useful. There would be an advantage to different mechanisms of action to existing IOP lowering treatments, which could be used to augment the effects of other IOP treatments. It would also be beneficial to have treatment methods that are easier to administer, such as orally with food or drink to avoid the issues associated with existing IOP treatments that are delivered topically to the eye and can be difficult and time consuming for patients to apply. Furthermore, it would be useful to have treatments that can be used prophylactically to prevent IOP elevation from occurring or lessen its severity. It would be advantageous if the treatment method could be used as a general prophylactic against aging changes in the eye and IOP elevation in the general population.

### SUMMARY

In one aspect, a method of treating or preventing elevated intraocular pressure in a subject is disclosed. In accordance with this aspect, a method of treating or preventing elevated intraocular pressure in a subject includes administering to the subject a therapeutically effective amount of nicotinamide or a derivative or analog of nicotinamide, wherein the nicotinamide or a derivative or analog of nicotinamide is administered to a human subject at a dose of from about 0.2 g/day to about 3 g/day.

In some embodiments, the subject has normal intraocular pressure.

In some embodiments, the subject has elevated intraocular pressure.

In some embodiments, the subject is at increased risk for developing glaucoma.

In some embodiments, administering to the subject a therapeutically effective amount nicotinamide, or a pharmaceutical composition thereof includes orally administering the nicotinamide, or a derivative or analog of nicotinamide or a pharmaceutical composition thereof.

In any one of the embodiments described herein, the method further comprises administering one or more additional therapeutic agents, or one or more pharmaceutical compositions thereof, to the subject.

In any one of the embodiments described herein, the one or more additional therapeutic agents are selected from the group consisting of an antioxidant agent, an anti-inflammatory agent, an agent that modulates metabolism, an agent that modulates the integrated stress response, an agent that modulates the unfolded protein response, an agent that modulates forms of autophagy, an agent that modulates the expression or activity of genes controlling or mediating antioxidant or other protective responses, a senolytic agent, an agent that modulates the mitochondria or mitophagy, an anti-aging agent, another agent that modulates intraocular pressure, a resilience-boosting agent, an antifibrotic agent, an agent that prevents epithelial mesenchymal transition or endothelial mesenchymal transition, a neuroprotective agent, a gene therapy agent, an agent that modulates vascular tone, an agent that modulates inflammatory and immune responses, an agent that modulates the TEK/angiopoietin system, any agents that increase the health, formation and functions of Schlemm's canal and/or other ocular drainage tissues, and a combination thereof.

In some embodiments, the additional therapeutic agent comprises a pyruvate compound, or a derivative or analog thereof, or a pharmaceutical composition thereof, to the subject.

In some embodiments, the pyruvate compound comprises ethyl pyruvate, or a derivative or analog thereof, or a pharmaceutical composition thereof, to the subject.

In some embodiments, the nicotinamide is administered at an animal equivalent dose corresponding to a mouse dose of from about 50 mg/kg/day to about 2500 mg/kg/day, more particularly from about 300 mg/kg/day to about 2000 mg/kg/day. In some embodiments, nicotinamide is administered to a human subject at a dose of from about 0.25 g/day to about 12 g/day, more particularly from about 1.5 g/day to about 10 g/day.

In some embodiments, the nicotinamide is administered at an animal equivalent dose corresponding to a mouse dose of from about 300 mg/kg/day to about 800 mg/kg/day. In some embodiments, the nicotinamide is administered to a human subject at a dose of from about 1.5 g/day to about 4 g/day.

In some embodiments, the nicotinamide is administered at an animal equivalent dose corresponding to a mouse dose of from about 1500 mg/kg/day to about 2500 mg/kg/day.

In some embodiments, the subject is a mammal.

In some embodiments, the subject is a human.

In some embodiments, the nicotinamide is administered for at least two weeks.

In accordance with another aspect, a method of treating or preventing elevated intraocular pressure in a subject includes administering to the subject a therapeutically effective amount nicotinamide, or a derivative or analog of nicotinamide or a pharmaceutical composition thereof, wherein the nicotinamide is administered at an animal equivalent dose corresponding to a mouse dose of from about 300 mg/kg/day to about 550 mg/kg/day, which corresponds to a human equivalent dose (HED) of from about 1.5 g/day to about 3 g/day for 60 kg human.

Any one of the embodiments disclosed herein may be properly combined with any other embodiment disclosed herein. The combination of any one of the embodiments disclosed herein with any other embodiments disclosed herein is expressly contemplated. Specifically, the selection of one or more embodiments for one substituent group can be properly combined with the selection of one or more particular embodiments for any other substituent group. Such combination can be made in any one or more embodiments of the application described herein or any formula described herein.

### DESCRIPTION OF THE DRAWINGS

The patent or application file contains at least one drawing executed in color. Copies of this patent or patent application publication with color drawing(s) will be provided by the Office upon request and payment of the necessary fee.

The application is described with reference to the following figures, which are presented for the purpose of illustration only and are not intended to be limiting. In the Drawings:

FIG. 1 is a graph of IOP for treated and untreated wildtype mice, according to one or more embodiments.

FIG. 2 is a graph showing facility for NAM treated and untreated 4-5 month old C57BL/6J (B6) mice, according to one or more embodiments.

FIG. 3 is a graph of IOP for NAM treated and untreated 12-14 months old DBA/2J-Gpnmb+ substrain mice, according to one or more embodiments.

FIG. 4 is a graph of IOP for NAM treated and untreated 4-6 months old mice with the Lmxlb^{V265D} mutation, according to one or more embodiments.

FIG. 5A is a graph of IOP for NAM treated and untreated 12-14 months old mice with the Lmx1b^{Q105X} mutation, according to one or more embodiments.

FIG. 5B is a graph of IOP for NAM treated and untreated 12 and 15 months old mice with the Lmx1b^{Q105X} mutation, according to one or more embodiments.

FIG. 6A shows UMAPs of limbal cells from WT and Lmx1b^{B265D/+} (V265) mutant mice (both genotypes B6 background).

FIG. 6B provides graphs showing Lmx1b expression across limbal cell types.

FIG. 6C provides charts showing that pathways are changed in all TM cell subtypes.

FIGS. 6D, 6E and 6F provide charts showing the top pathways that are significantly altered when comparing each indicated TM cell subtype across genotypes.

FIG. 7A provides representative photos of eyes from mice of the indicated genotypes and treatments (UNT=untreated, NAM=nicotinamide treated, 550 mg/kg/day provided in drinking water). NAM treatment substantially lessens anterior chamber deepening (ACD), a sensitive indicator of IOP elevation in mice. The WT and NAM-treated mutant eyes have very shallow anterior chambers while the untreated mutant eye has an obviously deepened chamber (arrowheads).

FIG. 7B is a bar chart showing distributions of anterior chamber depth based on previously defined scoring system (33462143). Groups compared by Fisher's exact test.

FIGS. 7C and 7D are Boxplots of IOP (interquartile range and median line) in WT and mutant eyes of both NAM treated and untreated groups. NAM treatment significantly lessens IOP elevation in mutants compared to untreated mutant controls. Groups compared by Two-way ANOVA followed by Tukey's Honestly significant difference. Geno=genotype.

### DETAILED DESCRIPTION OF THE INVENTION

In one aspect, a method of treating or preventing elevated intraocular pressure in a subject is disclosed. The method includes administering to the subject a therapeutically effective amount of nicotinamide, or a pharmaceutical composition thereof.

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

As used herein the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

An “effective amount”, “sufficient amount” or “therapeutically effective amount” as used herein is an amount of a compound that is sufficient to effect beneficial or desired results, including clinical results. As such, the effective amount may be sufficient, for example, to reduce or ameliorate the severity and/or duration and/or onset of elevated IOP. An effective amount also includes the amount of the compound that avoids or substantially attenuates undesirable side effects.

As used herein, the term “glaucoma” refers to an eye disease that results in damage to the retina and optic nerve and visual dysfunction or vision loss. Glaucoma occurs more commonly among older people. Vision loss from glaucoma is permanent and is irreversible.

As used herein, the term “normal intraocular pressure” (normal IOP) in humans refers to a human subject having an IOP value of 10 mmHg to 21 mmHg. Some individuals, however, may develop optic nerve damage despite a normal IOP (known as normal-tension glaucoma).

As used herein, the term “high intraocular pressure” (high IOP) in humans refers to a human subject having an IOP value greater than 21 mmHg (or 2.8 kPa). High IOP is known to be a risk factor for glaucoma. Some individuals, however, may have high IOP for years and never develop glaucoma or optic nerve damage.

As used herein, the term “preventing” or “prevention” with respect to, for example, neuronal damage or death in general or intraocular pressure in particular, refers to the ability of the compounds or agents of the present invention to confer neuroprotection, preferably before such damage, death, or disease occurs or to prevent a subject developing high intraocular pressure. Thus, prevention of high intraocular pressure includes avoiding the development of high intraocular pressure, reducing the risk or chance of eventually developing high intraocular pressure, delaying the onset or progression of high intraocular pressure, or reducing the severity of neuronal damage/extent of neuronal death/loss among a population of neurons should high intraocular pressure eventually develop.

As used herein and as well understood in the art, “treatment” is an approach for obtaining beneficial or desired results, including clinical results. Beneficial or desired clinical results may include, but are not limited to, alleviation or amelioration of one or more symptoms or conditions, diminution of extent of disease, a stabilized (i.e., not worsening) state of disease, preventing spread of disease, delay or slowing of disease progression, amelioration or palliation of the disease state and remission (whether partial or total), whether detectable or undetectable.

The present disclosure contemplates the use of derivatives or analogs of NAM as well. Derivatives or analogs of NAM may include, but are not limited to, nicotinic acid, nicotinamide riboside, nicotinamide mononucleotide and nicotinamide adenine dinucleotide (NAD+) and other nicotinoyl ribosides and nicotinamide riboside derivatives that promote the increase of intracellular levels of nicotinamide adenine dinucleotide (NAD+) in cells and tissues. Derivatives or analogs of NAM may also include, but are not limited to, conjugates with imaging agents, macromolecules, biomacromolecules, targeting agents, and isomers and combinations thereof; and pharmaceutically acceptable salts thereof.

In some embodiments, the method further comprises administering one or more additional therapeutic agents, or one or more pharmaceutical compositions thereof, to the subject. In some embodiments, the one or more additional therapeutic agents are selected from the group consisting of an antioxidant agent, an anti-inflammatory agent, an agent that modulates metabolism, an agent that modulates the integrated stress response, an agent that modulates the unfolded protein response, an agent that modulates forms of autophagy, an agent that modulates the expression or activity of genes controlling or mediating antioxidant or other protective responses, a senolytic agent, an agent that modulates the mitochondria or mitophagy, an anti-aging agent, another agent that modulates intraocular pressure, a resilience-boosting agent, an antifibrotic agent, an agent that prevents epithelial mesenchymal transition or endothelial mesenchymal transition, a neuroprotective agent, a gene therapy agent, and a combination thereof. In some embodiments, the agent that modulates metabolism reprograms or boosts metabolism. In some embodiments, the antioxidant agent reduces oxidative stress and boosts antioxidant control. In some embodiments, the gene therapy agent results in genome editing, genome reprogramming, epigenetic editing, epigenetic reprogramming, or a combination thereof. In some embodiments, the agent that prevents epithelial mesenchymal transition or endothelial mesenchymal transition is an anti-transforming growth factor-β (“TGFB”) or ligand trap molecule. In some embodiments, the one or more additional therapeutic agents are selected from the group consisting of pyrroloquinoline quinine, N-acetyl cysteine, a pyruvate compound and a combination thereof.

Examples of other agents that may be used in combination with NAM include, but are not limited to, saffron, berry extract or powder, antioxidants (e.g., bilberry, ergothioneine), flavonoids, anthocyanins, carotenoids, polyphenols, folate, xeoxanthine, polyamines (e.g., spermidine), Ginko biloba (glaucoma vitamin supplement (Glaucocetin), Co enzyme Q10, Vitamin A, Vitamin B12, Vitamin B50 complex, Vitamin C, Vitamin D, Vitamin E, betaine, choline, citicholine, Epigallocatechin-3-gallate (EGCG and its derivatives or analogs), compounds that increase activity of Sirts including Sirt3, lutein, xeozanthine, molecules that induce genes that protect from oxidative damage, pyruvate, dimethylfumarate and other forms of fumarate, and antifibrotic agents (such as anti-TGFBs, curcumin, blueberry, silymarin, coffee, vitamin C, E, and D, resveratrol, and quercetin). In accordance with some embodiments, the additional agent may be one or more Wnt signaling pathways activators, a β-catenin signaling modulator, MFSD2A, a derivative of MFSD2A or a modulator of MFSD2A. Examples of Wnt/beta-catenin agonists and molecules that increase MFSD2A are disclosed in more detail in WO 2025/019458 A2 to John et al. the contents of which are hereby incorporated by reference. In accordance with some embodiments, the additional agent may be a metabolism supporting molecule, such as, but not limited to: ketones, ketone bodies-metabolites used in energy metabolism (e.g., hydroxybutyrate, Beta-hydroxybutarate (BHB) and its salts, e.g., acetoacetate). The methods disclosed herein may also be combined with diets shown to help in lowering IOP such as, but not limited to, ketogenic diet and low carb diet. The methods disclosed herein may also be combined with glucagon like peptide (GLP-1) agonists including, but not limited to, Exenatide, Liraglutide and Semaglutide. Taurine and creatine can also be used in combination with the methods disclosed herein. Additional examples useful herein also include molecules and gene therapies that increase or induce NMNAT2 (nicotinamide mononucleotide adenosyl transferase) or other NMNATs, molecules or gene therapies that induce or otherwise increase signaling of the TEK/Angiopoietin system, any molecules that increase NAD, or insulin and its derivatives, agents or gene therapies that increase Ca²⁺/calmodulin-dependent protein kinase II (CAMKII) activity/signaling, treatments that increase CNTF, BNDF or other beneficial growth factors. This also includes without limitation encapsulated cells in implants that release the therapeutic agent(s) including these growth factors and/or proteins, peptides, antibodies, agonists, anti-inflammatory molecules, and metabolites. This also includes similar systems for ANGPT/TEK, insulin, etc. Additional examples that can be combined with NAM treatment include any IOP lowering medications e.g., Latanprost and other prostaglandin analogs. The method disclosed herein could also be combined with laser and/or other surgeries aimed at lowering IOP, with various implanted tubes, shunts and stents that lower IOP or long-term drug formulations and delivery devices. Biotin, hemp seeds/powder, long chain polyunsaturated fatty acids (PUFAS) (e.g., omega 3 fatty acids), spirulina and leafy green powder or extracts can also be combined with NAM treatment as disclosed herein. Derivatives and analogs of the compounds disclosed herein can also be used.

In some embodiments, the one or more additional therapeutic agents are administered together in a pharmaceutical composition with NAM. In other embodiments, the one or more additional therapeutic agents are administered separately from NAM. In still other embodiments, the subject is already being treated with the one or more additional therapeutic agents when the NAM is administered; or the subject is administered the one or more additional therapeutic agents after being administered the NAM.

In some embodiments, the NAM is administered in an amount sufficient to prevent or reduce elevated intraocular pressure, prevent or reduce one or more changes to the structure or function of one or more ocular tissues, prevent or reduce abnormal cell death, or a combination thereof. In some embodiments, the NAM is administered in an amount sufficient to prevent or reduce malformation or dysfunction of ocular drainage structures, prevent or reduce one or more developmental anomalies, prevent or reduce neural or non-neural cell degeneration, dysfunction, or death, or a combination thereof. In some embodiments, the NAM is administered in an amount sufficient to prevent or reduce one or more changes in the subject resulting from environmental exposure, disease, aging, metabolic anomaly, mitochondrial anomaly, genetic mutation, or a combination thereof. In some embodiments, the NAM is administered in an amount sufficient to prevent or reduce one or more developmental anomalies in the subject, such as, but not limited to, changes to anterior chamber depth, pupil abnormalities, iridocorneal adhesions, or a combination thereof. In some embodiments, the NAM is administered in an amount sufficient to prevent or reduce cell degeneration or dysfunction in the subject, such as, but not limited to, neurodegeneration and/or neural dysfunction in the subject's retinal ganglion cells. In some embodiments, the NAM is administered in an amount sufficient to prevent or reduce changes or dysfunction to the subject's ocular drainage structures, such as, but not limited to, Schlemm's canal, trabecular meshwork, or a combination thereof. In some embodiments, the NAM is administered in an amount sufficient to prevent or reduce a neurodegenerative disease or disorder in the subject, such as, but not limited to, a late onset neurodegeneration (e.g., Alzheimer's disease or Parkinson's disease). In some embodiments, the NAM is administered in an amount sufficient to prevent or reduce disease or disorder linked to one or more Lmxlb mutations, such as, but not limited to, a disease or disorder of the eye, brain, or kidney. In some embodiments, the NAM is administered in an amount sufficient to prevent or reduce a condition of aging in the subject, such as, but not limited to, age-related macular degeneration. In some embodiments, the NAM is administered in an amount sufficient to prevent or reduce a disorder of the retinal pigment epithelium in the subject.

In some embodiments, the amount of NAM sufficient to produce the effects described in the preceding paragraph will be the same when administered alone or in combination with one or more additional therapeutic agents. In some embodiments, the amount of NAM sufficient to produce the effects described in the preceding paragraph will be less when administered with one or more additional therapeutic agents than when administered alone.

In some embodiments, the subject is a mammal. In some embodiments, the subject is a mouse or rat. In some embodiments, the subject is of canine or equine origin. In some embodiments, the subject is a non-human primate. In some embodiments, the subject is a human.

In some embodiments, the subject has elevated IOP. In other embodiments, the subject is at risk, including, but not limited to, genetic risk, for developing elevated IOP. In other embodiments, the subject has elevated IOP and is at risk, including, but not limited to, genetic risk, for developing more serious variants or complications of the condition. In some embodiments, the subject has elevated IOP and is at risk, including, but not limited to, genetic risk, for developing additional conditions, which additional conditions may or may not be related to the original condition.

In another aspect, a method of reducing or preventing elevated intraocular pressure is described, the method comprising administering a therapeutically effective amount of NAM, or a pharmaceutical composition thereof, to the subject.

In another aspect, a method of reducing or preventing damage or dysfunction to a subject's eye tissues is described, the method comprising administering a therapeutically effective amount of NAM, or a pharmaceutical composition thereof, to the subject. In some cases, the subject is pregnant and the NAM is administered during prenatal care to protect the child from developing dysfunction/disease. In some cases, the subject is an infant and the NAM is administered through milk/formula supplementation or via the mother to protect the child from developing dysfunction/disease.

In another aspect, a method of reducing or preventing damage or dysfunction to tissues involved in ocular fluid drainage in a subject is described, the method comprising administering a therapeutically effective amount of NAM, or a pharmaceutical composition thereof, to the subject.

In another aspect, a method of reducing or preventing developmental malformations, disease dependent malformations or dysfunctions in a subject's eye is described, the method comprising administering a therapeutically effective amount of NAM, or a pharmaceutical composition thereof, to the subject.

In any one of the embodiments described herein, the method further comprises administering one or more additional therapeutic agents, or one or more pharmaceutical compositions thereof, to the subject.

In some embodiments, the NAM is administered in an amount sufficient to reduce intraocular pressure in the subject or to prevent the subject's intraocular pressure from increasing. In some embodiments, the subject has elevated intraocular pressure and the administration of NAM reduces the intraocular pressure or prevents the intraocular pressure from further increasing. In some embodiments, the subject does not have elevated intraocular pressure, and the administration of NAM prevents the subject's intraocular pressure from increasing.

In some embodiments, the NAM is administered in an amount sufficient to prevent or reduce changes in the subject's ocular drainage tissues. In some embodiments, the subject has the changes in ocular drainage tissues and the administration of NAM reduces the changes or prevents further changes. In other embodiments, the subject does not have the changes and the administration of NAM prevents the changes from developing. In some embodiments, the effects of NAM for preventing or reducing changes in the subject's ocular drainage tissues are related to its activity for modulation of intraocular pressure. In other embodiments, the effects of NAM for preventing or reducing changes in the subject's ocular drainage tissues are independent of its activity for modulating intraocular pressure.

In some embodiments, the NAM is administered in an amount sufficient to prevent or reduce one or more dysfunctions and/or developmental changes in the subject's eye. In some embodiments, the one or more dysfunctions and/or developmental changes are dysfunctions and/or malformations of the subject's ocular drainage tissues, including, but not limited to, Schlemm's canal and trabecular meshwork. In some embodiments, the one or more dysfunctions and/or developmental changes are anterior chamber depth, pupil abnormalities, and/or iridocorneal adhesions. In some embodiments, the subject has the dysfunctions and/or developmental changes and the administration of NAM reduces the developmental changes or prevents further developmental changes. In other embodiments, the subject does not have the dysfunctions and/or developmental changes and the administration of NAM prevents the developmental changes from developing. In some embodiments, the effects of NAM for preventing or reducing one or more dysfunctions and/or developmental changes in the subject's eye are related to its activity for modulation of intraocular pressure. In other embodiments, the effects of NAM for preventing or reducing one or more dysfunctions and/or developmental changes in the subject's eye are independent of its activity for modulating intraocular pressure.

In some embodiments, the disease or disorder of the eye is a neurodegenerative disease.

In some embodiments, the disease or disorder of the eye is a non-neurodegenerative disease.

In some embodiments, the disease or disorder of the eye is an age-related disease or disorder. In some embodiments, the disease of disorder of the eye is age-related macular degeneration. In some embodiments, age-related macular degeneration includes neurodegeneration. In some embodiments, age-related macular degeneration includes a retinal pigment epithelium (“RPE”) dysfunction. In some embodiments, age-related macular degeneration includes a vascular disease or condition.

In some embodiments, the prevention or reduction of the subject's elevated intraocular pressure prevents or treats a disease or disorder of the eye. In some embodiments, the disease or disorder of the eye comprises non-neural cell degeneration, dysfunction, and/or death. In some embodiments, the disease or disorder of the eye comprises neural cell degeneration, dysfunction, and/or death. In some embodiments, the disease or disorder of the eye is a neurodegenerative disease. In some embodiments, the disease or disorder of the eye is a non-neurodegenerative disease. In some embodiments, the disease or disorder of the eye is an age-related disease or disorder. In some embodiments, the disease of disorder of the eye is age-related macular degeneration.

In some embodiments, the prevention or reduction of the subject's elevated intraocular pressure prevents or treats neurodegeneration and/or neural dysfunction in the subject's retinal ganglion cells, changes in the subject's ocular drainage tissues, and/or developmental changes in the subject's eye.

In some embodiments, elevated IOP is a risk factor for a disease or disorder. In some embodiments, elevated IOP is a risk factor for a disease or disorder of the eye. In some embodiments, elevated IOP is a risk factor for a non-neurodegenerative or a neurodegenerative disease or disorder of the eye.

In some embodiments, elevated IOP is a risk factor for glaucoma. In some embodiments, elevated IOP is a risk factor for age-related macular degeneration. In some embodiments, elevated IOP is a risk factor for dysfunctions, malformations, and/or death of one or more cells, tissues, or structures in the eye.

In some embodiments, NAM protects ocular tissues from stresses due to environment, development, disease, and/or age (including, but not limited to, mitochondrial and metabolic abnormalities, Lmxlb mutation, or mutations in other genes impacting metabolism, and/or development).

In some embodiments, NAM protects ocular drainage structures from changes and cell death due to developmental conditions, genetic and epigenetic effects, and/or environmental and/or exposure effects. In some embodiments, NAM protects ocular drainage structures from malformation. In some embodiments, NAM protects from developmental anomalies of the eye. In some embodiments, NAM protects from developmental malformations that may have functional, cosmetic, and/or psychological impacts on the subject.

In some embodiments, NAM protects from cell death in ocular tissues and/or IOP elevation. In some embodiments, this cell death and IOP elevation is independent from glaucomatous neurodegeneration. Therefore, in certain embodiments, NAM protects from glaucomatous and other ocular degenerations. In some embodiments, NAM protects from effects of IOP elevating genes or genes/stresses impacting drainage tissue health and function.

In some embodiments, NAM protects from dysfunction and death of cells. In some embodiments, these cells may be nerve cells and/or neurons.

In some embodiments, NAM protects from diseases or disorders of the brain, including, but not limited to, late onset neurodegeneration (e.g., Parkinson's disease and Alzheimer's disease).

In some embodiments, NAM protects from diseases or disorders associated with aging.

In some embodiments, NAM, either alone or in combination with other therapeutic agents (e.g., a pyruvate compound), is an effective treatment for early-onset glaucoma with developmental abnormalities. In some embodiments, NAM, either alone or in combination with other therapeutic agents (e.g., a pyruvate compound), is an effective treatment for later-onset glaucomas.

In some embodiments, the inventors surprisingly found that NAM protects mice with a Lmxlb mutation. In some embodiments, Lmxlb mutations cause developmental defects, kidney disease, and/or glaucoma, with different ages of onset in mice versus humans. Therefore, in some embodiments, NAM treatment may protect from various Lmxlb-induced diseases.

In some embodiments, the inventors surprisingly found that NAM lessens IOP elevation in mouse models comprising a genetic model resulting in cell stress, developmental anomalies, cell death, and/or high IOP. In some embodiments, one or more of these factors may lead to glaucoma. In some embodiments, the mouse model is a Lmxlb mouse model.

In some embodiments, dosages of from about 50-5000, more particularly from about 200-2500, still more particularly from about 500-2000 mg/kg/day NAM lessen the severity of ocular developmental abnormalities and IOP elevation in mice. In some embodiments, combination of NAM and other agents capable of modulating metabolism (e.g., metabolic boosting agents) affords a higher degree of protection against the severity of ocular developmental abnormalities and IOP elevation in mice compared to NAM alone. For example, in some embodiments, combination of NAM and a metabolic boosting agent affords greater protection against developmental pupil abnormalities.

One of ordinary skill in the art can convert the dosages from one species to another using the teachings in Freireich et al., Quantitative comparison of toxicity of anticancer agents in mouse, rat, dog, monkey and man, Cancer Chemother Rep. 50(4):219-244, 1966 (incorporated herein by reference). This results in an animal equivalent dosage based on the mouse dosage. For example, a mouse dosage of 550 mg/kg NAM is equivalent to a dose of about 2.7 g/day for 60 kg person. In some embodiments, treatment is continued until a certain target IOP is obtained. In some cases, treatment is continued for an extended period of time, such as weeks, months or years to prevent development of elevated IOP.

In accordance with some embodiments, typical dosing may be once, twice, three or more times a day. Total daily dose may be administered once, or administered as two, three or more separate doses. For multiple dosing, each dose can be the same amount or different amounts. The pharmaceutical composition may be administered in the morning or evening. The pharmaceutical composition may be taken with or without meals.

In some embodiments, NAM enables use of lower dosages of other treatment modalities, such as, but not limited to, pyruvate compounds and other agents capable of modulating metabolism.

Pyruvate compounds, as used herein, include both the conjugate base pyruvate (CH₃COCOO—) and pyruvic acid (CH₃COCOOH). Pyruvate is the simplest of the alpha-keto acids, with a carboxylic acid and a ketone functional group, and is a key intermediate in several metabolic pathways.

In some embodiments, the pyruvate compound is a pharmaceutically acceptable salt of pyruvate. Pharmaceutically acceptable salt refers to the relatively non-toxic, inorganic and organic acid addition salts of the compounds. These salts can be prepared in situ during the final isolation and purification of the compound, or by separately reacting pyruvate with a suitable counterion and isolating the salt thus formed. Representative counterions include potassium, calcium, magnesium, ammonium, arginine, diethylamine, ethylenediamine, and piperazine salts, and the like. For example, in some embodiments, the pyruvate compound is selected from the group consisting of calcium pyruvate, potassium pyruvate, and magnesium pyruvate.

In some embodiments, the pyruvate compound is a pyruvate alkyl ester derivative. Pyruvate alkyl ester derivatives are forms of pyruvic acid in which an alkyl group is attached to the non-carbonyl oxygen of the carboxylic acid group. Particularly useful alkyl groups include alkyl groups having from 1 to 6 carbon atoms, alkyl groups having 2 carbon atoms are particularly useful (e.g., ethylpyruvate). Ethyl pyruvate has the chemical structure

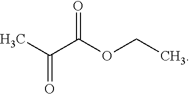

The present disclosure contemplates derivatives or analogs of ethyl pyruvate. Derivatives or analogs of ethyl pyruvate may include, but are not limited to, different ester chain lengths or substitutions, different substitutions on the acyl carbon, halogenated and isotopically-derived analogs, and isomers and combinations thereof, and pharmaceutically acceptable salts thereof. Derivatives or analogs of ethyl pyruvate may also include, but are not limited to, conjugates with imaging agents, macromolecules, biomacromolecules, targeting agents, and isomers and combinations thereof, and pharmaceutically acceptable salts thereof. Use of ethyl pyruvate or its derivatives and analogs in a pharmaceutical composition with one or more pharmaceutically acceptable excipients, and/or with one or more drug delivery or targeting vehicles, are also contemplated.

In some embodiments, a combination of ethyl pyruvate and NAM are particularly useful. In some embodiments, the dosage of ethyl pyruvate is from about 100-2000, more particularly from about 250-1000 mg/kg/day in mice. Human equivalent doses can be calculated as described in the literature.

### Pharmaceutical Compositions

This application also provides a pharmaceutical composition comprising at least one of the compounds as described herein or a pharmaceutically-acceptable salt thereof, and a pharmaceutically-acceptable carrier.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject pharmaceutical agent from one organ, or portion of the body, to another organ, or portion of the body. Each carrier must be “acceptable” in the sense of being compatible with the other ingredients of the formulation and pharmaceutically acceptable for the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as butylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

As set out above, certain embodiments of the present pharmaceutical agents may be provided in the form of pharmaceutically-acceptable salts. The term “pharmaceutically-acceptable salt”, in this respect, refers to the relatively non-toxic, inorganic and organic acid addition salts of compounds of the present application.

Wetting agents, emulsifiers and lubricants, such as sodium lauryl sulfate, magnesium stearate, and polyethylene oxide-polybutylene oxide copolymer as well as coloring agents, release agents, coating agents, sweetening, flavoring and perfuming agents, preservatives and antioxidants can also be present in the compositions.

Formulations of the present invention include those suitable for oral, nasal, topical (including buccal and sublingual), and/or parenteral administration. Particularly useful formulations include oral and direct to eye or locally around the eye formulations. The formulations may conveniently be presented in unit dosage form and may be prepared by any methods well known in the art of pharmacy. The amount of active ingredient which can be combined with a carrier material to produce a single dosage form will vary depending upon the host being treated and the particular mode of administration. The amount of active ingredient, which can be combined with a carrier material to produce a single dosage form will generally be that amount of the compound which produces a therapeutic effect. Generally, out of 100%, this amount will range from about 1% to about 99% of active ingredient, preferably from about 5% to about 70%, most preferably from about 10% to about 30%.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, sodium carbonate, and sodium starch glycolate; solution retarding agents, such as paraffin; absorption accelerators, such as quaternary ammonium compounds; wetting agents, such as, for example, cetyl alcohol, glycerol monostearate, and polyethylene oxide-polybutylene oxide copolymer; absorbents, such as kaolin and bentonite clay; lubricants, such a talc, calcium stearate, magnesium stearate, solid polyethylene glycols, sodium lauryl sulfate, and mixtures thereof, and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxybutylmethyl cellulose), lubricant, inert diluent, preservative, disintegrant (for example, sodium starch glycolate or cross-linked sodium carboxymethyl cellulose), surface-active or dispersing agent. Molded tablets, may be, made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein.

Liquid dosage forms for oral administration of the compounds disclosed herein include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isobutyl alcohol, ethyl carbonate, ethyl acetate, benzyl alcohol, benzyl benzoate, butylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. Additionally, cyclodextrins, e.g., hydroxybutyl-β-cyclodextrin, may be used to solubilize compounds.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives or buffers which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances.

Transdermal patches have the added advantage of providing controlled delivery of a compound of the present invention to the body. Such dosage forms can be made by dissolving or dispersing the pharmaceutical agents in the medium. Absorption enhancers can also be used to increase the flux of the pharmaceutical agents of the invention across the skin. The rate of such flux can be controlled, by either providing a rate controlling membrane or dispersing the compound in a polymer matrix or gel.

Ophthalmic formulations, eye ointments, powders, solutions and the like, are also contemplated as being within the scope of this disclosure. Implants or injectables may also be used to introduce the agent into the eye. Any of the excipients disclosed herein suitable for use in these applications can be incorporated into the pharmaceutical formulations.

Pharmaceutical compositions of this invention suitable for parenteral administration comprise one or more compounds disclosed herein in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

When the compounds of the present invention are administered as pharmaceuticals, to humans and animals, they can be given per se or as a pharmaceutical composition containing, for example, 0.1% to 99.5% (more preferably, 0.5% to 90%) of active ingredient in combination with a pharmaceutically acceptable carrier.

The representative examples which follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art. The following examples contain important additional information, exemplification, and guidance which can be adapted to the practice of this invention in its various embodiments and equivalents thereof.

### EXAMPLES

### Example 1: Lower Dose NAM Decreases IOP

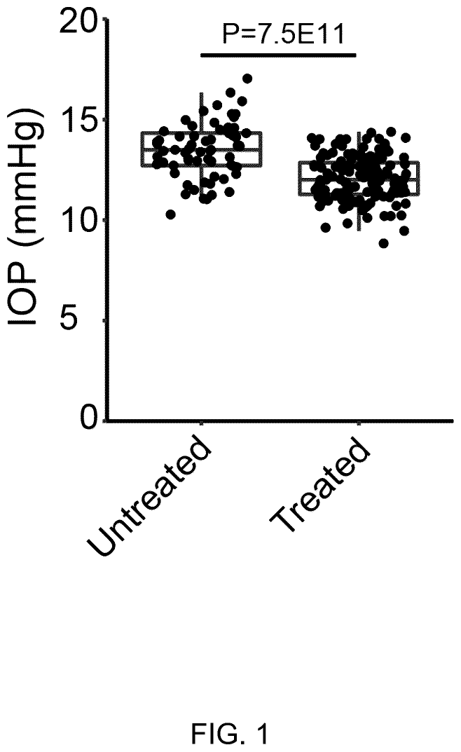

NAM at lower dose showed a decrease in IOP in mice (WT C57BL/6J (B6)), which were primarily 3 to 6 months old and of both sexes. The mice were treated with a relatively low dose of 500 mg/kg NAM in drinking water. Surprisingly, in view of previous studies, NAM lowered IOP to 89.6% of that of untreated mice as shown in FIG. 1.

A dose of about 2000 mg/kg NAM in mice is equivalent to about 9.8 g/day for a 60 kg human. A dose of about 550 mg/kg NAM in mice is equivalent to about 2.7 g/day for a 60 kg human.

### Example 2: NAM Treatment Increases Aqueous Humor Drainage (Outflow Facility)

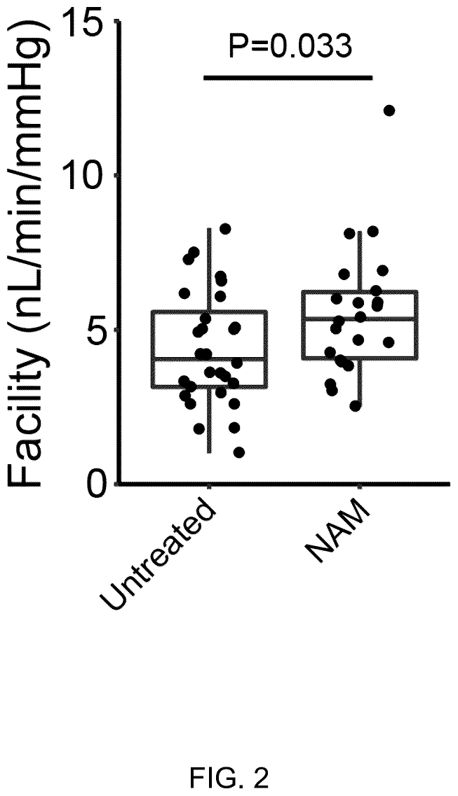

FIG. 2 shows that NAM treatment (550 mg/kg/day) raises outflow facility to 126% of untreated mice. The NAM was administered in drinking water to 4-5 month old C57BL/6J (B6) mice, half of each sex. Again, this is a surprising result, given previous studies, but shows that increased fluid drainage from the eye contributes to the IOP lowering. Although not wishing to be bound by theory, the NAM could be acting by both metabolically supporting drainage tissues and connected vasculature, as well as by dilating connected vasculature.

### Example 3: NAM Treatment Lessens IOP in Older Mice

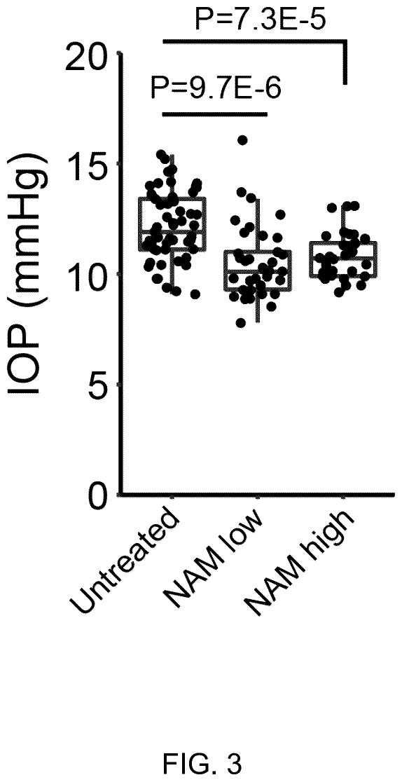

As shown in FIG. 3, both low dose (500 mg/kg) NAM treatment and high dose (2000 mg/kg) administered in water and food (12-14 months old DBA/2J-Gpnmb+ substrain, sex balanced and matched groups) resulted in a reduction in IOP. These mice are genetically distinct to B6 mice. Surprisingly, the low dose was as effective as the higher dose as shown in FIG. 3.

### Example 4: NAM Lowers IOP in Mutants with Earlier Onset High IOP and Glaucoma

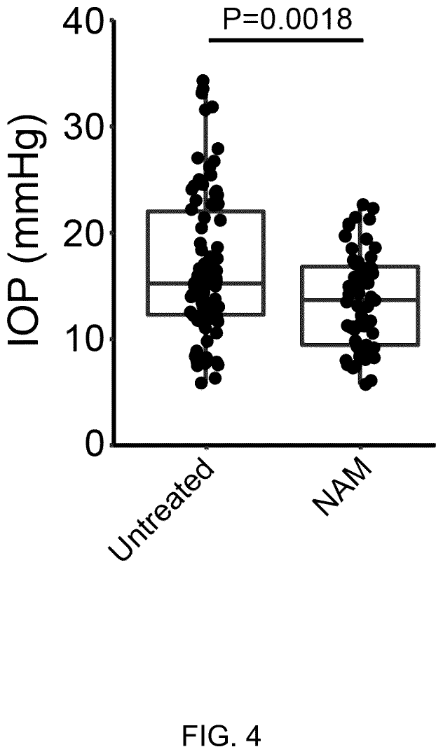

As shown in FIG. 4, treatment of mice harboring an early onset Lmx1b high IOP and developmental glaucoma model with NAM, unexpectedly lowered IOP. Mutations in Lmx1b cause high IOP and glaucoma in humans and mice. The Lmx1b^{V265D} mutation causes an earlier onset high IOP with severe glaucoma. Mutant mice treated with the lower dose of 500 mg/kg NAM administered in drinking water resulted in a reduction in IOP, which was 81% of that in untreated mutant mice. The mice were 4-6 months old, half of each sex. Over 93% of the treated mice had an IOP below 21 mmHg. IOP readings over 21 mmHg are commonly regarded as conferring higher glaucoma risk.

### Example 5: NAM Lowers IOP in Eyes with Later Onset High IOP and Glaucoma

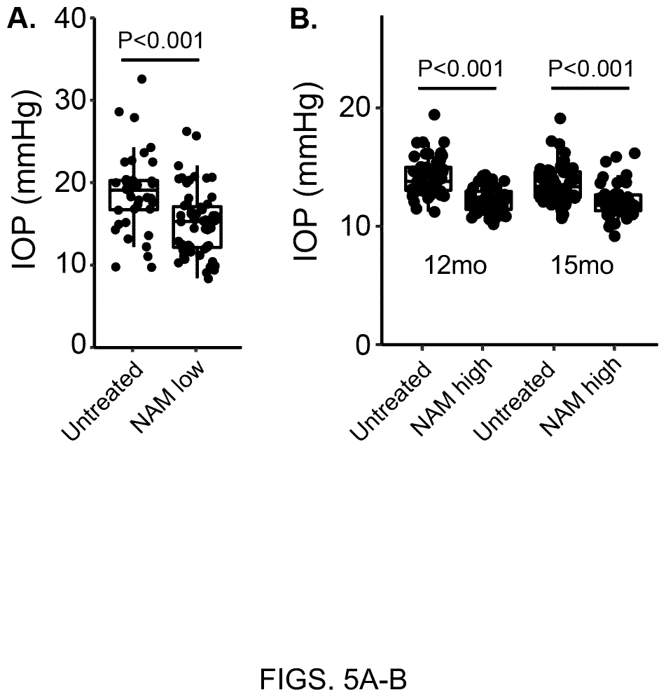

As shown in FIG. 5, treatment of mice harboring a late onset Lmx1b high IOP and developmental glaucoma model with NAM, unexpectedly lowered IOP. The Lmx1b^{Q105X} mutation causes a later onset high IOP and glaucoma phenotype that mimics common, age-dependent, human glaucoma. 12-14 months old mutant mice treated with NAM administered in drinking water resulted in a reduction in IOP as shown in FIG. 5A. NAM administered in drinking water and food also lessens IOP in genetically diverse Q105X mutants (diversity outbred (DO) cross, with a degree of diversity that mimics human populations) as shown in FIG. 5B.

NAM also lowered IOP in wild-type DO cross mice at 12 and 15 months of age (P=0.016 and P=0.005, respectively.

### Example 6: Metabolism-Supporting Treatment Lessens IOP Elevation in LHLx1b^{V265D} Mice

The trabecular meshwork (TM) is central to intraocular pressure (IOP) regulation and glaucoma. Through comprehensive multimodal analysis of mouse limbal cells (includes TM) using single-cell and single-nucleus RNA sequencing it was found that there are three TM cell subtypes with characteristic signature genes, which have been validated by immunofluorescence on tissue sections and whole-mounts. Distinctive signatures include increased expression of genes responsible for 1) extracellular matrix structure and metabolism (TM1 subtype), 2) secreted ligand signaling to support Schlemm's canal cells (TM2), and 3) contractile and mitochondrial/metabolic activity (TM3). ATAC-sequencing data identified transcription factors active in TM cells, including LMX1B. The focus was on LMX1B because mutations in LMXIB cause high IOP and glaucoma, LMX1B is emerging as a key transcription factor for normal mitochondrial function and its expression is much higher in TM3 cells compared to other limbal cells. To understand the role of LMX1B in TM function and disease, limbal cells from Lmx1b^{V265D/+} mutant mice were single-cell sequenced. In V265D+ mice, TM3 cells were uniquely affected by pronounced mitochondrial pathway changes; supporting a primary role of mitochondrial dysfunction in TM3 cells in initiating IOP elevation in LMX1B-induced glaucoma. Treatment with vitamin B₃ (nicotinamide) to enhance mitochondrial function/metabolism significantly protected Lmx1b mutant mice from IOP elevation.

The enrichment of mitochondrial metabolism pathways in TM3 cells emphasizes their energetic needs and their expected heightened susceptibility to genetic and environmental contexts that compromise metabolic functions. Genetic variation in mitochondrial/metabolic pathways impacts risk for IOP elevation and glaucoma.

The expression of genes implicated in elevated IOP and glaucoma were evaluated by genome-wide association studies (GWAS). TM1 and TM2 cells have similar expression levels of GWAS genes associated with IOP elevation and primary open-angle glaucoma (POAG) respectively. TM3 cells have slightly less expression of these GWAS genes compared to TM1 and TM2. To better understand the genes driving GWAS gene enrichment in different TM subtypes, pathways that are significantly enriched for GWAS genes were studied. TM1 cells are the most enriched for genes involved in ECM organization, cell-substrate adhesion, and growth factor stimulus. All 3 TM subtypes had similar expression of GWAS genes associated with hormone stimulus, Vegf signaling, and Rho kinase signaling. TM3 cells expressed actin binding-associated GWAS genes at higher expression levels and in more cells than did TM1 and TM2 cells. The same was true for lipid and carbohydrate mitochondrial metabolism pathway genes in TM3 cells versus TM1 and TM2. Variation in lipid and carbohydrate mitochondrial metabolism pathways is associated with IOP elevation and glaucoma.

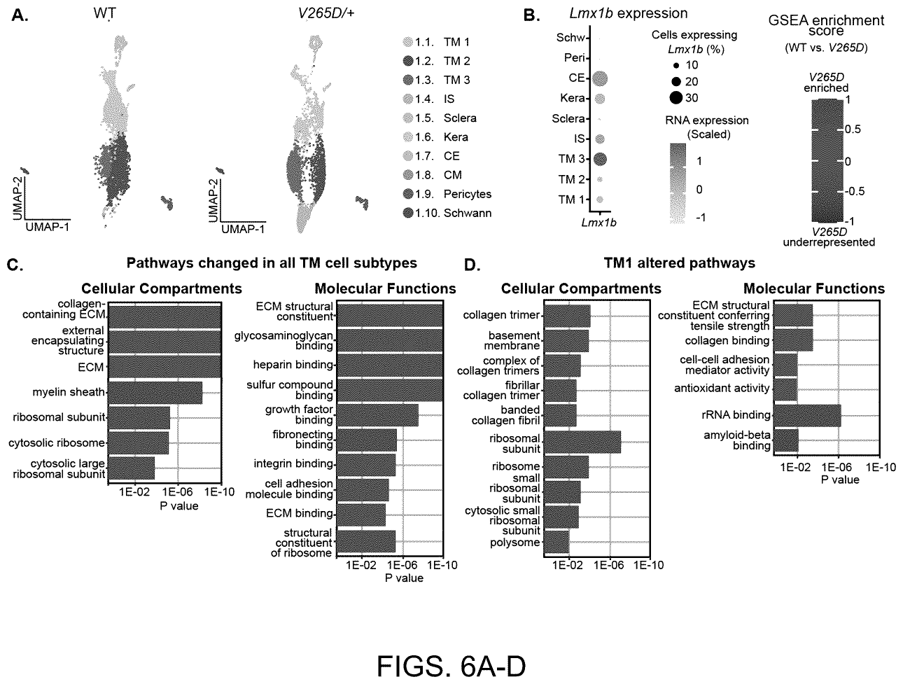

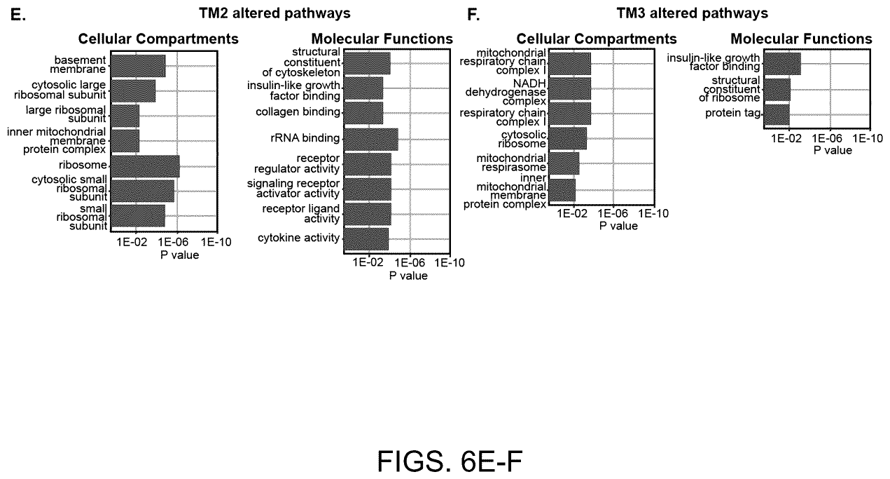

To better understand the function of Lmx1b in TM cells and how Lmx1b mutations lead to glaucoma, scRNA-seq was used to sequence limbal cells from mice with a dominant mutation in Lmx1b (Lmx1b^{V265D/+}) C57BL/6J mice with the V265D mutation develop early onset IOP elevation (Cross et al., 2014; Tolman et al., 2021). Gene expression in V265D mutant TM cells after integration was compared with wild-type data (all B6 background and all postnatal day 60 age). Lmx1b is minimally expressed in TM1 and TM2 but is highly expressed in TM3 cells (FIG. 6B). Next, DEGs and pathway differences between WT and V265D+ cells across each TM cell subtype were analyzed. Across all TM subtypes, various genes related to ECM function and growth factor signaling were downregulated in mutants, including glycosaminoglycans and insulin growth factor binding proteins (FIG. 6C). Pathways enriched in mutant TM cells were associated with ribosome and calcium signaling pathways. The differential responses to the Lmx1b mutation were then examined by analyzing pathways dysregulated in each TM subtype that were not common across all TM cells (FIGS. 6D-6F). V265D mutant TM1 cells had a downregulation of collagen synthesis/metabolism gene expression relative to WT cells, particularly in the production and turnover of fibril collagens. TM2 cells with Lmx1b mutations had upregulation of immune signaling pathways. TM3 cells exhibited perturbed mitochondrial metabolism, with a downregulation of genes in complex I and ATP synthase, and upregulation of genes involved in ubiquitin pathways. These results indicate altered mitochondrial function and proteostasis in TM3 cells.

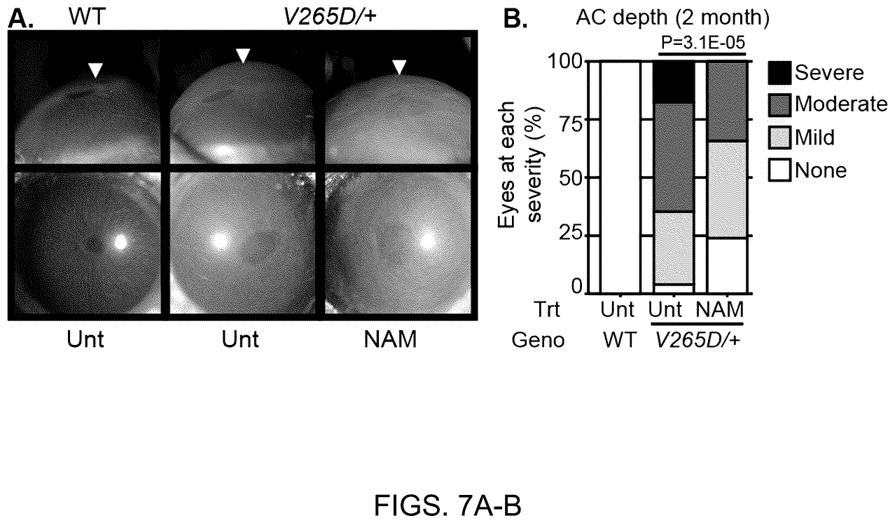

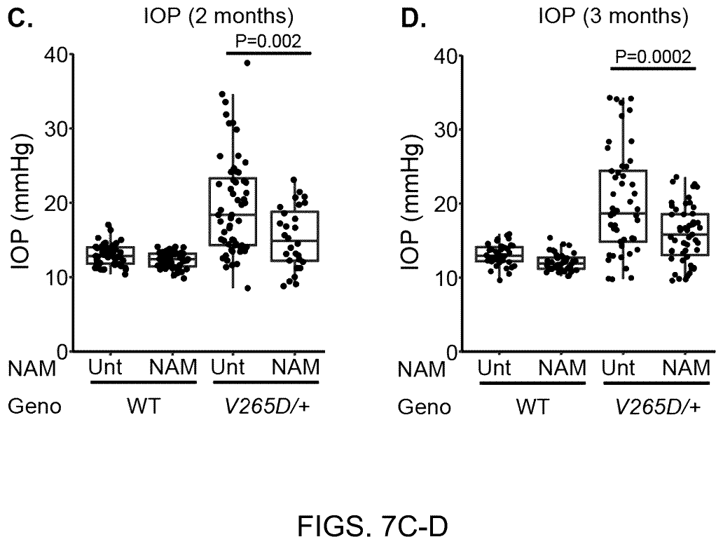

To test the bioinformatic predictions and role of TM3 in IOP control, treatment with NAM was evaluated to determine if it would protect mutants. Nicotinamide (NAM) boosts nicotinamide adenine dinucleotide (NAD) and supports healthy metabolism/mitochondrial function. Nicotinamide (NAM or niacinamide; Sigma-Aldrich, St. Louis, Missouri) was dissolved in the standard institutional drinking water to a dose of 550 mg/kg/day (low dose) based on the average volume mice consume. Untreated groups received the same drinking water without NAM. Water was changed once per week. Treatment was started at postnatal day 2. Births were checked daily between 9 am-12 pm to determine the pup's age. Results showed that NAM supplementation significantly protected against anterior chamber deepening (ACD, a symptom of IOP elevation) (FIGS. 7A-7B) and IOP elevation (FIG. 7C-7D). These data implicate metabolic health of TM3 cells as important for normal IOP control and support NAM as a treatment to prevent IOP elevation in LMX1B-mediated IOP elevation and glaucoma.

The present application provides a comprehensive multimodal analysis of mouse limbal cells that focuses on the TM. Three TM cell subtypes with unique marker panels that can be used for their identification across different strains, laboratories, and processing methods have been identified. All three TM subtypes are enriched in ECM synthesis and ECM metabolism compared to other limbal subtypes. However, there is also compartmentalized enrichment of some processes to specific subtypes. For example, ECM production and turnover, secreted ligand signaling, debris removal, immune surveillance, actin-associated cell contraction and increased metabolic gene activity are enriched in specific cell-subtypes. Using ATAC-seq data, key transcription factors controlling gene expression in TM cells, including LMX1B, were discovered. Importantly, LMX1B is implicated in metabolic control in TM cells. More specifically, Lmx1b was found to be most active in TM3 cells, while mutation of Lmx1b induces prominent mitochondrial defects in TM3 cells. The present disclosure also shows that therapeutically targeting TM3 dysfunction with a mitochondrial/metabolism supporting nutrient (nicotinamide) protects from IOP elevation in the Lmx1b glaucoma model. Together these data provide a wealth of new molecular information about TM cells.

TM3 cells also have a location and nuclear morphology resembling beam cells with enriched localization in the inner, anterior TM. TM3 cell-enriched processes include actin binding, and modulation of the actomyosin system, which is known to modulate outflow resistance and IOP. As such, TM3 cells express a large percentage of glaucoma-associated genes involved in actin binding at a high level. Importantly, TM3 cells are enriched for various mitochondrial metabolic and antioxidant pathways, including those associated with IOP elevation and glaucoma. Inhibiting metabolic pathways alters AqH outflow resistance. Since maintaining contractile tone is an energy-intensive process, this could explain the enrichment of energy metabolism pathways in TM3 cells compared to other TM cells.

In addition to being enriched in mitochondrial/energy metabolism processes, TM3 cells express Lmx1b at significantly higher levels than both the other TM cell subtypes and other limbal cells. Importantly, in heterozygous mutant V265D+ mice, TM3 cells had pronounced gene expression changes that implicate mitochondrial dysfunction and oxidative stress, but that were absent or much lower in other cells including TM1 and TM2. Together, these data suggest that mutation of Lmx1b has a primary effect on metabolism and oxidative stress in TM3 cells that subsequently leads to IOP elevation and then glaucoma. Homozygous conditional knockout of both Lmx1b and Lmx1a as well as siRNA knockdown of Lmx1b alone impact mitochondrial functions in neurons and may contribute to Parkinson's disease. The data disclosed herein extend these findings by showing that inheritance of a single dominant mutation in Lmx1b similarly affects TM3 cells. In addition to modulating mitochondria, LMX1B recently was identified as transcription factor that regulates responses to cell stress. This includes promotion of autophagy under stress conditions to enable recycling of cellular resources to maintain cellular functions and enable survival. Thus, mutation of Lmx1b may also result in a deficiency of the beneficial autophagic response to stress. Dysfunctional autophagy may further prolong and exacerbate TM cell stress, resulting in more extensive depletion of cellular resources, exacerbated cellular dysfunction, and IOP elevation. Other consequences of the Lmx1b mutation that were evident in the V265D+ cells may also contribute to IOP elevation, including the overexpression of genes associated with ribosomes and calcium signaling, and depletion of genes involved with ECM synthesis and metabolism. However, these processes were not limited to TM3 cells or even to cell types that express detectable Lmx1b-expressing cells, suggesting that they are secondary damaging processes that are subsequent to the initiating, Lmx1b-induced, metabolic disturbances and cell stress in TM3 cells. Thus, mitochondrial abnormalities in TM3 cells are thought to be of primary importance to IOP elevation in V265D+ glaucomatous mice.

### Nicotinamide Protects Against IOP Elevation in Lnx1b^{V265D}D-Mutant Mice

Based on the findings in TM3 cells, mitochondrial and metabolic disturbances were tested to determine if they are important in IOP elevation by administering nicotinamide (NAM). NAM is a form of vitamin B3 that boosts NAD⁺ levels, promotes mitochondrial health and energy metabolism, relieves oxidative stress and thus promotes cellular resilience Consistent with the concept of primary metabolic dysfunction in TM3 cells, NAM treatment strongly protected V265D+ mice from IOP elevation. Thus, treatments that support normal metabolism may protect from IOP elevation in individuals with LMXIB variants including both developmental glaucoma and POAG patients. As NAM and other NAD⁺ boosting treatments may be expected to have a double benefit for patients. As mitochondrial defects are becoming broadly associated with glaucoma risk and progression, these treatments could have wide-ranging potential to complement and augment existing IOP-lowering therapeutics through a completely distinct mechanism.

The results disclosed herein provide a thorough molecular characterization of mouse TM cells, providing much needed molecular information on subtype specializations in regulating IOP and the roles of specific subtypes in glaucoma. They provide a new foundation to guide development of new glaucoma treatments, with particular emphasis on metabolism supporting treatments.

What is claimed is:

1. A method of treating or preventing elevated intraocular pressure in a subject, the method comprising administering to the subject a therapeutically effective amount of nicotinamide or a derivative or analog of nicotinamide, wherein the nicotinamide or a derivative or analog of nicotinamide is administered to a human subject at a dose of from about 0.2 g/day to about 3 g/day.

2. The method of claim 1, wherein the subject has normal intraocular pressure.

3. The method of claim 1, wherein the subject has elevated intraocular pressure.

4. The method of claim 1, wherein the subject is at increased risk for developing glaucoma.

5. The method of claim 1, wherein the nicotinamide or a derivative or analog of nicotinamide, is orally administered.

6. The method of claim 1, further comprising administering to the subject an additional therapeutic agent.

7. The method of claim 6, wherein the one or more additional therapeutic agents are selected from the group consisting of an antioxidant agent, an anti-inflammatory agent, an agent that modulates metabolism, an agent that modulates the integrated stress response, an agent that modulates the unfolded protein response, an agent that modulates forms of autophagy, an agent that modulates the expression or activity of genes controlling or mediating antioxidant or other protective responses, a senolytic agent, an agent that modulates the mitochondria or mitophagy, an anti-aging agent, another agent that modulates intraocular pressure, a resilience-boosting agent, an antifibrotic agent, an agent that prevents epithelial mesenchymal transition or endothelial mesenchymal transition, a neuroprotective agent, a gene therapy agent, and a combination thereof.

8. The method of claim 7, wherein the additional therapeutic agent comprises a pyruvate compound.

9. The method of claim 8, wherein the pyruvate compound comprises ethyl pyruvate.

10. The method of claim 9, wherein the ethyl pyruvate is administered at a dose of from about 0.5 g/day to about 10 g/day.

11. The method of claim 1, wherein the nicotinamide or a derivative or analog of nicotinamide is administered at a dose of from about 1.5 g/day to about 3 g/day.

12. The method of claim 11, wherein the subject is not afflicted with glaucoma.

13. The method of claim 1, wherein the nicotinamide or a derivative or analog of nicotinamide is administered for at least two weeks.

14. The method of claim 1, wherein the derivative or analog of nicotinamide is selected from the group consisting of nicotinic acid, nicotinamide riboside, nicotinamide mononucleotide, nicotinamide adenine dinucleotide and combinations thereof.

15. The method of claim 14, wherein the derivative or analog of nicotinamide is nicotinamide riboside.

16. A method of reducing or preventing damage or dysfunction to tissues involved in ocular fluid drainage in a subject, the method comprising administering a therapeutically effective amount of nicotinamide or a derivative or analog of nicotinamide, wherein the subject is not afflicted with glaucoma.

17. The method of claim 16, wherein the derivative or analog of nicotinamide is selected from the group consisting of nicotinic acid, nicotinamide riboside, nicotinamide mononucleotide, nicotinamide adenine dinucleotide and combinations thereof.

18. The method of claim 17, wherein the derivative or analog of nicotinamide is nicotinamide riboside.

19. The method of claim 16, wherein the nicotinamide or a derivative or analog of nicotinamide is administered at a dose of from about 1.5 g/day to about 3 g/day.
